(19) United States
(12) Patent Application Publication
(10) Pub. No.: US 2021/0369844 A1
(43) Pub. Date: Dec. 2, 2021

(54) ANTIMICROBIAL APPARATUS COMPRISING FIELD-ELECTRIC NANOPARTICLES (FENPS) AND METHOD THEREOF

(71) Applicant: Ping Liang, Newport Coast, CA (US)

(72) Inventor: Ping Liang, Newport Coast, CA (US)

(21) Appl. No.: 17/445,086

(22) Filed: Aug. 14, 2021

Related U.S. Application Data
(63) Continuation-in-part of application No. 16/379,792, filed on Apr. 10, 2019, now Pat. No. 11,103,720.
Application No. 16/379,792 is a continuation-in-part of application No. 15/578,218, filed on Nov. 29, 2017, now Pat. No. 10,335,487.
(60) Provisional application No. 62/181,936, filed on Jun. 19, 2015.

(51) Int. Cl. A61K 41/00; A61K 9/51; A61K 47/69; A61M 37/00

(52) U.S. Cl. CPC A61K 41/0047; A61M 37/00; A61K 47/6929; A61K 9/5115

(57) ABSTRACT

The present invention provides an apparatus for targeting and disrupting, deactivating or destroying microorganisms (e.g. viruses, bacteria, fungus or diseased cells). The apparatus includes Field-Electric Nano-Particles coated, conjugated or functionalized with one or more guiding agents such as antibodies or proteins that target a type of bacteria, fungus, virus or diseased cells; a delivery module to deliver such nanoparticles into a subject's body, and an external energy field generation module. The nanoparticles, when subject to the applied external energy field, generate an electric field or pulses of electric field localized to the targeted bacteria, fungus or virus to disrupt, deactivate or destroy the targeted bacteria, fungus, viruses, or diseased cells.

### CROSS-REFERENCE TO RELATED APPLICATIONS

This non-provisional application is a Continuation-in-Part of U.S. patent application Ser. No. 16/379,792 filed Apr. 10, 2019, which is a Continuation-in-Part of U.S. patent application Ser. No. 15/578,218 filed Nov. 29, 2017 and granted as U.S. patent Ser. No. 10/335,487 on Jul. 2, 2019, which is a National Stage Application of International Application No. PCT/US2016/037619 filed Jun. 15, 2016, which claims the benefit of U.S. Provisional Application No. 62/181,936 filed on Jun. 19, 2015, the entire disclosures of which three prior applications are incorporated herein by reference.

### FIELD OF THE INVENTION

The present invention generally relates to an antimicrobial apparatus comprising Field-Electric Nanoparticles (FENPs) and a method thereof. Although the invention will be illustrated, explained and exemplified by Guiding-Agent-Conjugated Magneto-Electric Nanoparticles (GAC-MENPs), it should be appreciated that the present invention can also be applied to other types or functionalized FENPs, for example, Ultrasonic-Electric Nano-Particles (UENPs), Drug-Coated FENPs (DC-FENPs), and the like. In some embodiments, the present invention relates to an apparatus for targeting and disrupting or destroying biological organisms such as viruses, bacteria. fungus, or diseased cells, using localized electric field generated by types of nanoparticles converting energy from an applied external field, and more specifically, using Field-Electric Nano-Particles (FENPs) or Magneto-Electric Nano-Particles (MENPs).

### BACKGROUND OF THE INVENTION

There exists an urgent need for a readily available treatment to disrupt or destroy viruses, bacteria or fungus, especially novel viruses for which there is no known effective drugs and vaccines, e.g., Ebola or SAS-COV-2, and antibiotic-resistant bacteria and fungus infections of the skin or organ that are not treatable by known drugs. An apparatus that can effectively treat a large variety of novel viruses, drug-resistant bacteria, and fungus infections without effective treatment such as mucormycosis black fungus infection of the lungs in COVID-19 patients, is highly desired but lacking. It is known from basic biology that functions of virus, bacteria, fungus and diseased cells can be affected by an applied electric field or current. However, there is no prior art that can effectively generate electric field or current that is localized to the targeted virus, bacteria or fungus or diseased cells and strong enough to disrupt, deactivate or destroy them, and not damage surrounding healthy cells. There is no prior art that possess the functions of the embodiments presented in this invention.

Advantageously, the present invention provides an antimicrobial apparatus comprising Field-Electric Nanoparticles (FENPs) that can meet the need as described above.

### SUMMARY OF THE INVENTION

One aspect of the present invention provides an antimicrobial apparatus for disrupting or destroying microorganism such as bacteria, fungus, viruses, or diseased cells. The apparatus includes (i) Guiding-Agent-Conjugated Field-Electric Nanoparticles (GAC-FENPs) that are produced by coating, binding, functionalizing or conjugating to Field-Electric Nano-Particles (FENPs) with one or more guiding agent that specifically targets or binds to a targeted bacteria, fungus or virus or diseased cells; (ii) a delivery module that delivers the GAC-FENPs into a subject's body; and (iii) an external energy field generation module that generates and applies an external energy field to act on the GAC-FENPs after the GAC-FENPs are in the proximity of, or bind to, the targeted bacteria, fungus, viruses, or diseased cells and produce a localized electric field or pulses of electric field that acts or act on the targeted bacteria, fungus, viruses, or diseased cells to disrupt or deactivate the function of, or destroy, the targeted bacteria, fungus, viruses, or diseased cells.

The above features and advantages and other features and advantages of the present invention are readily apparent from the following detailed description of the best modes for carrying out the invention when taken in connection with the accompanying drawings.

### BRIEF DESCRIPTION OF THE SEVERAL VIEWS OF THE DRAWINGS

The present invention is illustrated by way of example, and not by way of limitation, in the figures of the accompanying drawings and in which like reference numerals refer to similar elements. All the figures are schematic and generally only show parts which are necessary in order to elucidate the invention. For simplicity and clarity of illustration, elements shown in the figures and discussed below have not necessarily been drawn to scale. Well-known structures and devices are shown in simplified form, omitted, or merely suggested, in order to avoid unnecessarily obscuring the present invention.

FIG. 1A schematically illustrates an antimicrobial apparatus for disrupting or destroying microorganism such as bacteria, fungus, viruses, or diseased cells according to various embodiments of the present invention.

FIG. 1B shows the components of an embodiment of an apparatus for disrupting or destroying bacteria, fungus, viruses or diseased cells using GAC-FENPs, a delivery module and an external energy field generation module.

FIG. 2 shows a preferred process of applying the apparatus for disrupting or destroying bacteria, fungus, viruses, or diseased cells using GAC-FENPs, a delivery module and an external energy field.

FIG. 3 shows another preferred process of applying the apparatus for disrupting or destroying bacteria, fungus, viruses, or diseased cells using GAC-FENPs, a delivery module, a navigation module and an external energy field generation module.

FIG. 4 shows another preferred process of applying the apparatus for disrupting or destroying bacteria, fungus, viruses, or diseased cells using FENPs coated, conjugated or functionalized with both one or more guiding agents and one or more chemicals, molecules or drugs, a delivery module, a navigation module and an external energy field generation module.

### DETAILED DESCRIPTION OF THE PREFERRED EMBODIMENT

In the following description, for the purposes of explanation, numerous specific details are set forth in order to provide a thorough understanding of the present invention. It is apparent, however, to one skilled in the art that the present invention may be practiced without these specific details or with an equivalent arrangement.

Where a numerical range is disclosed herein, unless otherwise specified, such range is continuous, inclusive of both the minimum and maximum values of the range as well as every value between such minimum and maximum values. Still further, where a range refers to integers, only the integers from the minimum value to and including the maximum value of such range are included. In addition, where multiple ranges are provided to describe a feature or characteristic, such ranges can be combined.

It is also to be understood that the terminology used herein is for the purpose of describing particular embodiments only, and is not intended to limit the scope of the invention. For example, when an element is referred to as being “on”, “connected to”, or “coupled to” another element, it can be directly on, connected or coupled to the other element or intervening elements may be present. In contrast, when an element is referred to as being “directly on”, “directly connected to”, or “directly coupled to” another element, there are no intervening elements present.

Throughout the specification and claims, the following terms take the meanings explicitly associated herein, unless the context clearly dictates otherwise. The phrase “in one embodiment” does not necessarily refer to the same embodiment, although it may. Furthermore, the phrase “in another embodiment” does not necessarily refer to a different embodiment, although it may. Thus, as described below, various embodiments of the invention may be readily combined without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The term “diseased cells” in this invention may mean cells that cause diseases, deficient cells, malfunctioning cells, or cells damaged or undesirably modified or deformed by a disease or diseasecausing factor(s). Examples include sickle cells, cancer cells, white blood cells (lymphocytes) that make autoantibodies that cause Systemic Lupus Erythematosus, abnormal tertiary lymphoid structures in organs targeted by autoimmune attacks.

Without being bound by any particular theory, it is believed that a sufficient strong electric field or current applied to biological organisms including viruses, fungus, bacteria and diseased cells can disrupt their functions and/or ability to replicate, destroy them. It is a form of electrocution. The challenge is when the viruses, fungus or bacteria or diseased cells are inside a biological body, how to deliver a strong electric field or current to act on the viruses, bacteria and fungus or diseased cells without electrocute normal tissues and cells. There is no prior art that can effectively generate electric field or current that is localized to the targeted virus, bacteria or fungus or diseased cells and strong enough to disrupt, deactivate or destroy them, and not damage surrounding healthy cells. This invention provides a solution to solve this problem.

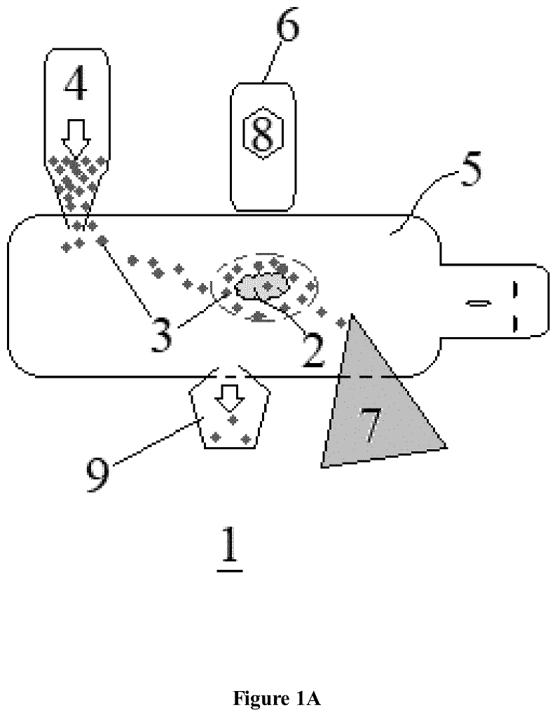

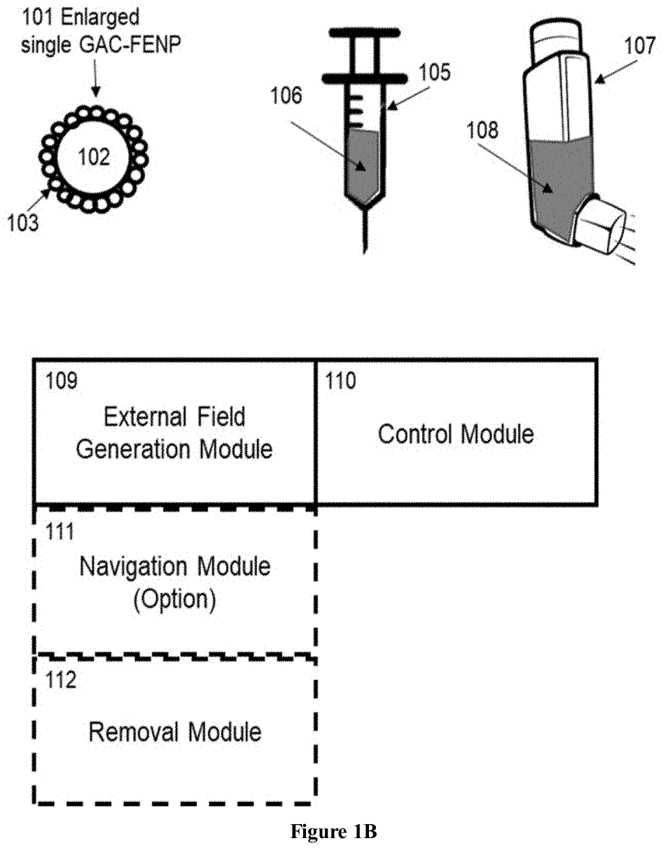

With reference to FIG. 1A, an antimicrobial apparatus 1 is designed for disrupting or destroying microorganism 2 such as bacteria, fungus, viruses, or diseased cells. Guiding-Agent-Conjugated Field-Electric Nanoparticles (GAC-FENPs) 3 are produced by coating, binding, functionalizing or conjugating to Field-Electric Nano-Particles (FENPs) with one or more guiding agent that specifically targets or binds to a targeted bacteria, fungus or virus, or diseased cells 2.

Examples of the guiding agents include, but are not limited to immunoglobulin, antibody, antibody mimetic, ligands, cell adhesion peptides, or other molecules or proteins that have an affinity to or bind to a targeted bacteria, fungus, virus, or diseased cells 2. In other embodiments, the FENPs' surfaces are first coated, functionalized or conjugated with one or more layer of material, molecule or compound to modify its surface property before being coated, conjugating or functionalized with the guiding agent that targets or binds to a targeted bacterium, fungus, diseased cells or virus to produce the GAC-FENPs 3. Sometimes, the FENPs may be coated, bounded, conjugated or functionalized both with one or more drugs, molecules or chemicals that modify, affect or neutralize a targeted bacteria, fungus, virus or diseased cells and one or more guiding agents that target a bacteria, fungus or virus, or diseased cells 2 to produce Guiding-Agent-Conjugated Drug-Coated FENPs (GAC-DC-FENPs) 3.

It is also contemplated that the FENPs are coated, bounded, conjugated or functionalized with one or more drugs, molecules or chemicals (instead of said one or more guiding agents as described above) that modify, affect or neutralize a targeted bacteria, fungus, virus, or diseased cells 2 to produce Drug-Coated FENPs (DC-FENPs) 3.

A delivery module 4 delivers the GAC-FENPs 3 into a subject's body 5. After the GAC-FENPs 3 are in the proximity of, or bind to, the targeted bacteria, fungus, viruses, or diseased cells 2, an external energy field generation module 6 generates and applies an external energy field to act on the GAC-FENPs 3 and produce a localized electric field or pulses of electric field on the GAC-FENPs that acts or act on the targeted bacteria, fungus, viruses, or diseased cells 2 to disrupt or deactivate the function of, or destroy, the targeted bacteria, fungus, viruses, or diseased cells 2. In preferred embodiments of invention, the external energy field generation module 6 is configured to adjust the characteristics of the generated external field so that it causes electric field on the GAC-FENPs bound to the targeted bacteria, fungus, viruses, or diseased cells 2 to disrupt or deactivate or damage the targeted bacteria, fungus, viruses, or diseased cells 2 but it does not cause other GAC-FENPs 3 that still remain in the body but unbound to targeted bacteria, fungus, viruses, or diseased cells 2 to harm healthy or untargeted cells.

The external field energy field generation module 6 may include a control module 8 that programs or controls the external field energy field generation module 6 to produce or apply external energy field with a desired spatial, temporal or spatiotemporal pattern to yield a desired treatment effect or plan.

In some embodiments, the external energy field generation module 6 may be a magnetic field or electromagnetic field generation module, the Field-Electric Nano-Particles (FENPs) may be Magneto-Electric Nano-Particles (MENPs), and the GAC-FENPs may be Guiding-Agent-Conjugated Magneto-Electric Nanoparticles (GAC-MENPs). Given that, the external energy field generation module 6 will generate a magnetic or an electromagnetic field and apply the magnetic or the electromagnetic field to act on the GAC-MENPs 3. In preferred embodiments, MENPs may each have a core-shell structure. For example, MENPs may contain a composition of CoFe₂O₄—BaTiO₃, NiFe₂O₄—BaTiO₃, and/or Fe₃O₄—BaTiO₃. The external energy field generation module 6 may include one or more stationary or moving permanent magnets, one or more electromagnets driven by varying electric currents, or any combination thereof.

In various exemplary embodiments, the magnetic or electromagnetic field generation module 6 may employ one or more of the following mechanisms to disrupt or destroy the bacteria, fungus or virus 2 after the GAC-FENPs 3 have bound to or have electroporated inside the targeted bacteria, fungus or virus 2: (1) applying an alternating external magnetic field to induce an alternating electric field on the GAC-MENPs 3 whereas the strength and frequency of the field is selected such that it disrupts the functions of the bacteria, fungus, viruses, or diseased cells 2; (2) applying an alternating external magnetic field to induce mechanical motions of the GAC-MENPs 3 to disrupt or deactivate the functions or to physically damage the bacteria, fungus, viruses, or diseased cells 2, and the mechanical motions may include linear motion, slicing, collisions or vibrations, or combinations thereof; (3) applying an alternating external magnetic field of a resonant frequency that is modified by the interaction of GAC-MENPs 3 with the nano-environment to induce a ferromagnetic resonance in the GAC-MENPs 3 to disrupt or deactivate or shut down the operation of the bacteria, fungus, viruses, or diseased cells 2; and (4) applying an external constant magnetic field to modify the resonant frequency of the GAC-MENPs 3, and thereafter applying an alternating external magnetic field at a frequency that causes a ferromagnetic resonance in the GAC-MENPs 3 at or near the modified resonant frequency to selectively disrupt or deactivate the function of the bacteria, fungus, viruses, or diseased cells.

Typically, the external energy field generation module 6 generates an electromagnetic field in one or more of the following frequency ranges from 1 Hz to 10 trillion Hz (THz), such as audio frequency, radio frequency, optical frequency, near or far infrared frequency. A navigation module 7, when the FENPs are MENPs, comprising one or more permanent magnets or electro-magnets may be placed, injected or implanted in or near a site with a high concentration of the (or a concentrated) targeted virus, bacteria or fungus or diseased cells 2 to attract or guide the GAC-MENPs 3 to said site. In certain general embodiments, the antimicrobial apparatus 1 as shown in FIG. 1A includes a navigation module 7 that generates an external energy field and uses it to guide, attract and/or enhance accumulation of the GAC-FENPs into a site with a high concentration of targeted viruses, bacteria or fungus, or diseased cells.

In other embodiments of the invention, the external energy field generation module 6 may be an ultrasound energy field generation module. Accordingly, the Field-Electric Nano-Particles (FENPs) comprise Ultrasonic-Electric Nano-Particles (UENPs), and the GAC-FENPs 3 comprise Guiding-Agent-Conjugated Ultrasonic-Electric Nanoparticles (GAC-UENPs) 3. Such an external energy field generation module 6 generates an ultrasound energy field and applies the ultrasound energy field to act on the GAC-UENPs 3.

In some embodiments as shown in FIG. 1A, the delivery module 4 may be manufactured as an injection syringe for injecting a solution of the GAC-FENPs 3 into a subject's body 5, an aerosolization device for inhalation of the GAC-FENPs 3 into a subject's body 5, a pressured or diffusion delivery device for infuse the GAC-FENPs 3 into a subject's body 5 across the skin, or an encapsulation device to produce GAC-FENPs 3 containing capsules or pills for ingesting the GAC-FENPs 3 into a subject's body 5.

The antimicrobial apparatus 1 as shown in FIG. 1A may be used for localized treatment of a targeted site. The external energy field generation module 6 is configured to generate and apply a first external energy field to lead to a higher concentration of GAC-FENPs 3 at and around a targeted site or in an organ or body part with a high concentration of the targeted bacteria, fungus, viruses, or diseased cells 2 before it generates and applies a second external energy field to cause the GAC-FENPs 3 to generate an electric field or pulses of electric field to disrupt the function or destroy the targeted bacteria, fungus, viruses, or diseased cells 2.

The external energy field generation module 6 may also be configured to generate and apply an energy field after a waiting period to give the subject's body 5 sufficient time to excrete most or all of the free GAC-FENPs 3 that did not bind to targeted bacteria, fungus, viruses, or diseased cells out of the body, and/or for the GAC-FENPs 3 to target or bind to the targeted viruses, bacteria or fungus 2 before generating and applying the external energy field to cause damages to the targeted bacteria, fungus, viruses, or diseased cells 2.

The antimicrobial apparatus 1 as shown in FIG. 1A may include a removal module 9 configured for removing the FENPs from the subject's body 5. For example, the removal module 9 may be configured to produce a sufficiently strong energy field and apply it to areas of the subject's body 5 that contain GAC-FENPs 3 to attract the GAC-FENPs 3 to facilitate the removal of the GAC-FENPs 3 from the subject's body 5. In a specific embodiment, the removal module 9 is configured to apply an extraction solution that contains agents that binds to the GAC-FENPs 3; and attract the GAC-FENPs 3 bound with the agents to the surface of the subject's body 5 for removal.

A specific embodiment of the present invention is an apparatus as illustrated in FIG. 1B for disrupting or destroying bacteria, fungus, viruses, or diseased cells. The apparatus includes (i) Guiding-Agent-Conjugated Field-Electric Nanoparticles (GAC-FENPs) 101 that are produced by coating, binding, functionalizing or conjugating to Field-Electric Nano-Particles (FENPs) 102 one or more guiding agent 103 that specifically targets or binds to a targeted bacterium or virus; (ii) a delivery module, e.g., a syringe 105 for injection of a solution 106 of GAC-FENPs or an inhaler 107 of a powder, vapor or liquid form 108 of GAC-FENPs, or a cream or patch to be applied to the surface of one or more skin areas, that delivers the GAC-FENPs into a subject's body; and (iii) an external energy field generation module 109 that generates and applies an external energy field to act on the GAC-FENPs after the GAC-FENPs are in the proximity of or bind to the targeted bacteria, fungus, viruses, or diseased cells to produce localized electric field or pulses of electric field that acts on the targeted bacteria, fungus, viruses, or diseased cells to disrupt or deactivate the function of or destroy the targeted bacteria, fungus, viruses, or diseased cells. The external field energy field generation module 109 further includes (or works with) a control module 110 that programs or controls the external field energy field generation module 109 to produce or apply a desired spatial, temporal or spatiotemporal pattern of the external energy filed to yield the desired treatment effect or plan.

Furthermore, the one or more guiding agent comprises one or more of immunoglobulin, antibody, antibody mimetic, cell adhesion peptides, ligands, or other molecules or proteins that have an affinity to or bind to a targeted bacterium or virus to produce the GAC-FENPs. In particular, conjugation of antibodies to the FENPs equips the conjugated nanoparticles with the specific and selective recognition ability of the antibodies to antigens, thus increases the accumulation of the conjugated nanoparticles to the targeted viruses, bacteria or fungus, enabling the GAC-FENPs to deliver electric field localized to the targeted viruses, bacteria or fungus. There are well established processes for conjugating guiding agents such as antibodies and proteins to nanoparticles. They can be used or adapted to apply in this invention in the fabrication of the GAC-FENPs.

A guiding agent such as antibody targeting a specific virus does not need to be neutralizing because the ability to disrupt, deactivate or destroy the virus is provided by the local electric field produced by the GAC-FENPs when an external energy field is applied. This makes it much easier and quicker to identify an antibody or other guiding agent when a novel virus such as SAS-COV-2, or dangerous bacteria such as carbapenem-resistant Enterobacteriaceae, or fungus such as black fungus mucormycetes emerges or start to spread to threaten public health.

In one embodiment, Magneto-Electric Nano-Particles (MENPs) are used as the FENPs. MENPs can couple the energy from an externally applied magnetic field or electromagnetic field to produce an electric field on the nanoparticles. In this case, the GAC-FENPs are referred to as GAC-MENPs, and the external energy field generation module is a magnetic or electromagnetic field generation module that generates and applies a magnetic or electromagnetic field to act on the GAC-MENPs. One type of MENPs has a magnetic core, a piezoelectric shell and an interface between the core and shell. When a magnetic field is applied to MENPs, the particles produce an electric polarization due to the magnetic-electric coupling property of the core and shell at their interface. This electric polarization can produce a local electric field of 5,000 to 10,000V/cm depending on the strength of the applied field. Such local electric field can be strong enough to disrupt or destroy targeted viruses, bacteria or fungus when the MENPs are brought close to or bound to the target, e.g., attached to them or located tens of nanometer (nm) away or closer.

One type of MENPs is made with a basic structure of CoFe₂O₄—BaTiO₃ coreshell. One embodiment uses 30-nm MENPs synthesized from the following steps: 1) 0.058 g of Co(NO₃)₂.6H₂O and 0.16 g of Fe(NO₃)₃.9H₂O are dissolved in 15 mL of deionized (DI) water; 2) 5 mL of aqueous solution containing 0.9 g of sodium borohydride and 0.2 g of polyvinylpyrrolidone is added at 120° C. for 12 hours to obtain CoFe₂O₄ nanoparticles; 3) BaTiO₃ precursor solution is prepared by adding 30 mL of DI water containing 0.029 g of BaCO₃ and 0.1 g of citric acid to 30 mL ethanolic solution containing 1 g of citric acid and 0.048 mL of titanium (IV) isopropoxide; 4) As-prepared CoFe₂O₄ nanoparticles (0.1 g) is added to the 60 mL of BaTiO₃ precursor solution and sonicated for 120 min; 5) The resulted dispersed nanoparticles is dried on hot plate at 60° C. for 12 hours, while stirring at 200 rpm; 6) The obtained powder is heated at 780° C. for 5 hour in a box-furnace and cooled at 52° C. min⁻¹ to obtain 30 nm-sized CoFe₂O₄.BaTiO₃ core-shell MENPs. In another embodiment, the nano-particles are further surface functionalized by a compound, e.g., a 2-nm thick coating of glycerol mono-oleate (GMO) using the following steps: (i) GMO-MENPs is prepared by incubating 0.1 mg of GMO with 5 mg of MENPs in 5 mL of PBS (pH 7.4) buffer for 12 hours; to achieve uniform surface modification, the solution is slowly agitated during incubation; (ii) The solution is centrifuged at 20000 rpm for 20 min at 10° C. to remove excess GMO; (iii) The obtained pellet is re-suspended in ethyl acetate:acetone (70:30) solution and re-centrifuged three times to obtain GMO-MENPs. (iv) Surface-modified MENPs were lyophilized and stored at 4° C. until further use. The MENPs can then be conjugated with a guiding agent, for example an antibody that targets a specific virus, or a protein that attaches to the membrane of a specific bacterium. Other types of MENPs have a core-shell structure or composition of CoFe₂O4-BaTiO₃, NiFe₂O4-BaTiO₃, or Fe₃O₄—BaTiO₃, and can be fabricated using similar or modified processes.

Shape, size, external energy field coupling efficiency and other properties of the FENPs are important for the embodiments of this invention. One embodiment for fabricating FENPs with a wide range of properties comprises first depositing a thin film with the required properties via sputter deposition, evaporation, or another deposition technique, and then using ion beam proximity lithography (IBL) or imprint or another advanced lithography method to “cut” the thin films into AC-MENPs of desired shapes and sizes.

In one embodiment, antibodies are conjugated to the compound-coated MENPs, e.g., GMO-MENPs, e.g., covalently attaching antibodies onto the compound-coated MENPs' or GMO-MENPs' surface. In one example, to covalently attach antibodies to compound-coated MENPs, the nanoparticle surface is preliminarily functionalized. In the case of GMO-MENPs, 1 mg of GMO-MENPs are added to a solution of the PBS buffer (pH 7.4). To this solution, a solution of N-(3-Dimethylaminopropyl)-N′-ethyl-carbodiimide hydrochloride and N-hydroxysuccinimide, at 1 mg/ml concentration in the PBS buffer are added. The solution is incubated for 4 hours at room temperature while being stirred slowly. Then, the sample is centrifuged at 14,000 rpm for 10 minutes at 10° C. and the pellet is washed three times with 1 ml of the PBS buffer (pH 7.4). To bind antibodies to the functionalized MENPs, a solution of the antibodies (1 mg/ml) is added to the pellet along with a solution of the PBS buffer. The solution is incubated for 2 hours while being rotated slowly and kept further at 4° C. overnight. The solution is centrifuged at 14,000 rpm for 10 minutes at 10° C. The pellet was washed twice with 1 ml of the PBS buffer to remove any excess antibody.

In general, FENPs' surfaces can be first coated, functionalized or conjugated with one or more layer of material, molecule or compound to modify its surface property before being coated, conjugating or functionalized with the guiding agent that targets or binds to a targeted bacterium or virus to produce the GAC-FENPs.

In the embodiments using MENPs, the external energy field generation module may comprise one or more stationary or moving permanent magnets, and/or one or more electromagnets driven by varying electric currents. Furthermore, the magnetic or electromagnetic field generation module may further apply one or more of the following mechanisms to disrupt or destroy the bacteria, fungus or virus after the GAC-FENPs have bound to or have electroporated inside the targeted bacteria, fungus or virus:

applying an alternating external magnetic field to induce an alternating electric field on the GAC-MENPs whereas the strength and frequency of the field is selected such that it disrupts the functions of the bacteria, fungus, viruses, or diseased cells;

applying an alternating external magnetic field to induce mechanical motions of the GAC-MENPs to disrupt or deactivate the functions or to physically damage the bacteria, fungus, viruses, or diseased cells; where the mechanical motions may include linear motion, slicing, collisions or vibrations, or combinations thereof;

applying an alternating external magnetic field of a resonant frequency that is modified by the interaction of GAC-MENPs with the nano- or micro-environment to induce a ferromagnetic resonance in the GAC-MENPs to disrupt or deactivate or shut down the operation of the bacteria, fungus, viruses, or diseased cells; or,

applying an external constant magnetic field to modify the resonant frequency of the GAC-MENPs, and thereafter applying an alternating external magnetic field at a frequency that causes a ferromagnetic resonance in the GAC-MENPs at or near the modified resonant frequency to selectively disrupt or deactivate the function of the bacteria, fungus, viruses, or diseased cells.

The MENPs may be fabricated to couple external energy field in frequency ranges from several Hz to THz, e.g., audio, radio, optical, near or far infrared frequency energy waves, to generate electric field. Thus, the external energy field generation module 109 may be designed and built to generate an electromagnetic field in one or more of the following frequency ranges from several Hz to THz: audio, radio, optical, near or far infrared.

In one embodiment, a type of MENPs that work in the radio frequency range, e.g., from MHz to multi-GHz are used. These are referred to as Radio-Frequency-Electric Nano-Particles (RFENPs) which couple the energy from an externally applied radio-frequency electromagnetic field to produce an electric field, and they may be used as the FENPs to produce Guiding-Agent-Conjugated Radio-Frequency-Electric Nanoparticles (GAC-RFENPs). The external energy field generation module is a magnetic or electromagnetic field generation module that generates and applies a radio-frequency electromagnetic field to act on the GAC-RFENPs.

In yet another embodiment, a type of MENPs that work in the optical, infrared or near infrared frequency range are used, and the external energy field generation module is a magnetic or electromagnetic field generation module that generates and applies an optical, infrared or near infrared frequency electromagnetic field to act on the GAC-MENPs.

To bring the GAC-FENPs, e.g., GAC-MENPS, close to sites with a high concentration of the targeted virus or bacterium (referred hereafter to as targeted sites), one embodiment further comprises using an external energy field to guide and/or enhance the accumulation of the GAC-FENPs into such sites. In the case of GAC-MENPs, one or more permanent magnets or electro-magnets can be placed, injected or implanted in or near a site with a high concentration of the targeted virus, bacteria or fungus, whereas the magnet(s) serves to attract or guide the MENPs to and increase the accumulation of GAC-MENPs at the targeted site. The general embodiments of FENPs further comprises a navigation module 111, as shown in FIG. 1B, that generates and uses an external energy field to guide, attract and/or enhance the accumulation of the GAC-FENPs into a targeted site with a high concentration of targeted viruses, bacteria or fungus. The navigation module 111 may be either an independent module or an attachment or component of the external field generation module 109, especially when the energy field used to navigate the GAC-FENPs are the same type of field used to generate electric field on the GAC-FENPs. In such embodiments, the same energy field generation mechanism in the external field generation module 109 is used but the control module 110 in 109 programs or controls 109 to produce the spatial, temporal or spatiotemporal pattern of the generated energy field to achieve the goal of navigating or guiding the GAC-FENPs to targeted sites.

In another embodiment, a changing field is used instead of a constant one to generate a varying or pulsating force to push GAC-FENPs towards a targeted site. In the case of GAC-MENPs, the changing magnetic field can be generated by driving a periodic or irregular alternating current through one or more electro-magnets, e.g., a sine or square wave current, or by varying the position of one or more permanent magnets, e.g., rolling, rotating or moving back and forth of permanent magnets. The period, pattern, magnitude and/or direction of the alternating magnetic field can be changed to achieve desired movements of the MENPs.

Constant (DC) external field and changing (AC) external field play different roles in the embodiments using GAC-FENPs for targeting and destroying viruses, bacteria or fungus. In cases where both a DC magnetic field and an AC magnetic field are desired at the same time, one embodiment is an apparatus formed into a shape and dimension to fit a targeted site or volume that is made of or using permanent magnetic material and with one or more, e.g., an array of, electromagnets embedded in the apparatus to generate a DC magnetic field and AC magnetic field simultaneously.

In one embodiment, Ultrasonic-Electric Nano-Particles (UENPs) that couple the energy from an externally applied ultrasound field to produce an electric field are used as the FENPs to produce Guiding-Agent-Conjugated Ultrasonic-Electric Nanoparticles (GAC-UENPs), and the external energy field generation module generates an ultrasound energy field and applies the ultrasonic field to act on the GAC-UENPs. The UENPs can be produced using a nano- or micron-sized particles using piezoelectric materials and a structure that generates strains or deformations on the piezoelectric material structure when subject to an ultrasonic energy field, thus producing an electric field on the particles as a function of the strength and frequency of the applied ultrasonic field energy.

The delivery module of the apparatus may comprise an injection syringe for injecting a solution of the GAC-FENPs into a subject's body, an aerosolization device for inhalation of the GAC-FENPs into a subject's body, a cream or patch to be applied to topically to the surface of the skin, a pressured or diffusion delivery device for infuse the GAC-FENPs into a subject's body across the skin, or an encapsulation device to produce GAC-FENPs containing capsules or pills for ingesting the GAC-FENPs into a subject's body.

Furthermore, the external energy field generation module adjusts the characteristics of the generated external field, to generate an external energy field on the GAC-FENPs that is sufficient to disrupt or deactivate the function or destroy the targeted bacteria, fungus, viruses, or diseased cells while at the same time, avoiding or minimize damages to surrounding normal, healthy or non-targeted cells. In the embodiments of MENPs, the magnetic or electromagnetic field generation module further chooses the characteristics of the magnetic or electromagnetic field so that it causes electric field on the GAC-MENPs bound to the targeted bacteria, fungus, viruses, or diseased cells to disrupt or deactivate or damage the targeted bacteria, fungus, viruses, or diseased cells but does not cause other GAC-MENPs that still remain in the body and unbound to targeted bacteria, fungus, viruses, or diseased cells to harm healthy or untargeted cells.

When the apparatus is used for localized treatment of a targeted site, wherein the external energy field generation module further comprising a navigation module 111 as an attachment or a component or function to generate and apply a first external energy field to lead to a higher concentration of GAC-FENPs at and around a targeted site or in an organ or body part with a high concentration of the targeted bacteria, fungus, viruses, or diseased cells before generating and applying a second external energy field to cause the GAC-FENPs to generate an electric field or pulses of electric field to disrupt the function or destroy the targeted bacteria, fungus, viruses, or diseased cells.

In one embodiment, the external energy field generation module generates and applies an energy field after a waiting period to give the body sufficient time to excrete out of the body most or all of the free GAC-FENPs that did not bind to targeted bacteria, fungus, viruses, or diseased cells, and/or for the GAC-FENPs to target or bind to the targeted viruses, bacteria or fungus before generating and applying the external energy field to cause damages to the targeted bacteria, fungus, viruses, or diseased cells.

Furthermore, the profile or characteristics of the external energy field, e.g., frequency, pattern, magnitude and direction, can be adjusted to generate an electric field sufficient to disrupt the function or destroy the targeted bacteria, fungus, viruses, or diseased cells. At the same time, the characteristics of the external energy field, e.g., magnetic or electromagnetic field, may be chosen to avoid or minimize damages to surrounding normal cells.

In another embodiment, the GAC-FENPs are coated, bounded, conjugated or functionalized with one or more drugs, molecules or chemicals that modifies, affects or neutralizes a targeted bacterium or virus to produce Drug-Coated FENPs (DC-FENPs). The DC-FENPs are also coated, bounded, conjugated or functionalized with a guiding agent such as antibodies or proteins that target a type of bacteria, fungus or virus to produce Guide-Agent-Conjugated Drug-Coated FENPs (GAC-DC-FENPs).

In one embodiment, after the AC-MENPs have bound to or have electroporated inside the targeted bacteria, fungus or virus, one or more of the following mechanisms is applied to destroy the bacteria, fungus or virus:

(A). Apply an external magnetic field to generate strong enough electric field on the AC-MENPs to destroy the bacteria, fungus or virus, e.g., local electric fields of ≥1000 V/cm, which can be attained a few nanometers away from AC-MENPs via the application of an external magnetic field of ≥100 Oe;

(B). Apply an alternating external magnetic field to induce an alternating electric field on the AC-MENPs whereas the strength and frequency of the field is selected such that it disrupts the functions of the bacteria, fungus, viruses, or diseased cells, thus causes them to die off;

(C). Apply an alternating external magnetic field to generate heat on the AC-MENPs to destroy the bacteria, fungus, viruses, or diseased cells whereas the strength and frequency of the field is selected to generate sufficient heat to destroy the bacteria, fungus, viruses, or diseased cells without harming surrounding cells or tissues; and

(D). Apply an alternating external magnetic field to induce mechanical motions of the AC-MENPs to disrupt or deactivate the functions or to physically damage the bacteria, fungus, viruses, or diseased cells, causing them to die off; where the mechanical motions may include linear motion, slicing, collisions or vibrations, or combinations thereof.

In another embodiment, a ferromagnetic resonance strongly dependent on the interaction of AC-MENPs with its nano-environment (in the proximity of a few nanometers away from the nano-particles) is used to selectively disrupt or deactivate or shut down the operation of the bacteria, fungus, viruses, or diseased cells, when AC-MENPs are bound to or have electroporated the bacteria, fungus, viruses, or diseased cells. The ferromagnetic resonance of AC-MENPs depends on the saturation magnetization, which in turn, because of the magneto-electric (ME) effect, strongly depends on the electric fields that are associated with the interaction of AC-MENPs with the nano-environment. As the nano-environment changes, so does the saturation magnetization and consequently the ferromagnetic resonance frequency(ies). This resonant frequency or set of resonant frequencies can be varied in a wide range by varying intrinsic properties, e.g. the magneto-crystalline anisotropy energy and the exchange coupling constant, or extrinsic properties, e.g. the shape-induced anisotropy energy. In addition, the resonant frequency(ies) can be controlled by application of an external DC magnetic field. By specifically selecting the resonant frequencies, certain functions of cancer cells can be shut down with a relatively high specificity on demand. For example, the microtubules responsible for cancer cell proliferation could be remotely controlled via ferromagnetic resonance of the AC-MENPs. Namely, the resonant frequency of AC-MENPs in the proximity (of 2 nm) of the microtubules changes because of the changes in the saturation magnetization. The saturation magnetization change is due to the ME effect caused by the interaction of the AC-MENPs and the microtubules. An external AC magnetic field at the new modified resonant frequency can then by applied to disrupt or deactivate or cause damages to the bacteria, fungus, viruses, or diseased cells.

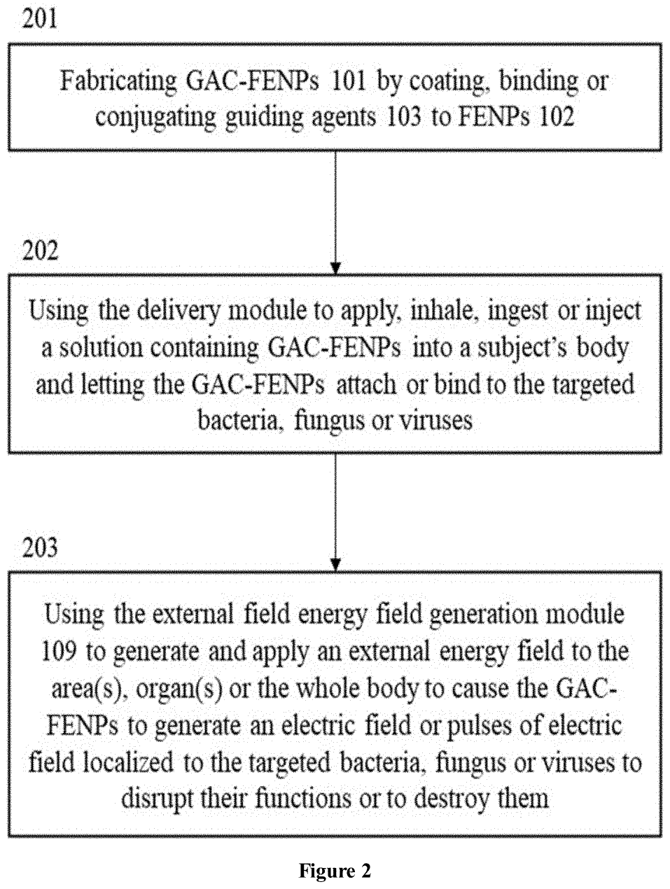

The apparatus of this invention provides a novel treatment for disrupting or destroying of bacteria, fungus, viruses, or diseased cells that is non-toxic or low-toxic. The steps of one preferred embodiment of applying the apparatus for the intended purpose is shown in FIG. 2, comprising:

201: Fabricating GAC-FENPs 101 by coating, binding or conjugating guiding agents 103, such as antibodies or proteins, that have an affinity to or bind to a type of bacteria or virus to FENPs 102;

202: Using the delivery module, e.g., 105 or 107, to apply, inhale, ingest or inject a solution containing GAC-FENPs into a subject's body and letting the GAC-FENPs attach or bind to the targeted bacteria, fungus, viruses, or diseased cells; and

203: Using the external field energy field generation module 109 to generate and apply an external energy field, e.g., a magnetic, electromagnetic or ultrasonic energy field to the area(s), organ(s) or the whole body to cause the GAC-FENPs to generate an electric field or pulses of electric field localized to the targeted bacteria, fungus, viruses, or diseased cells to disrupt their functions or to destroy them.

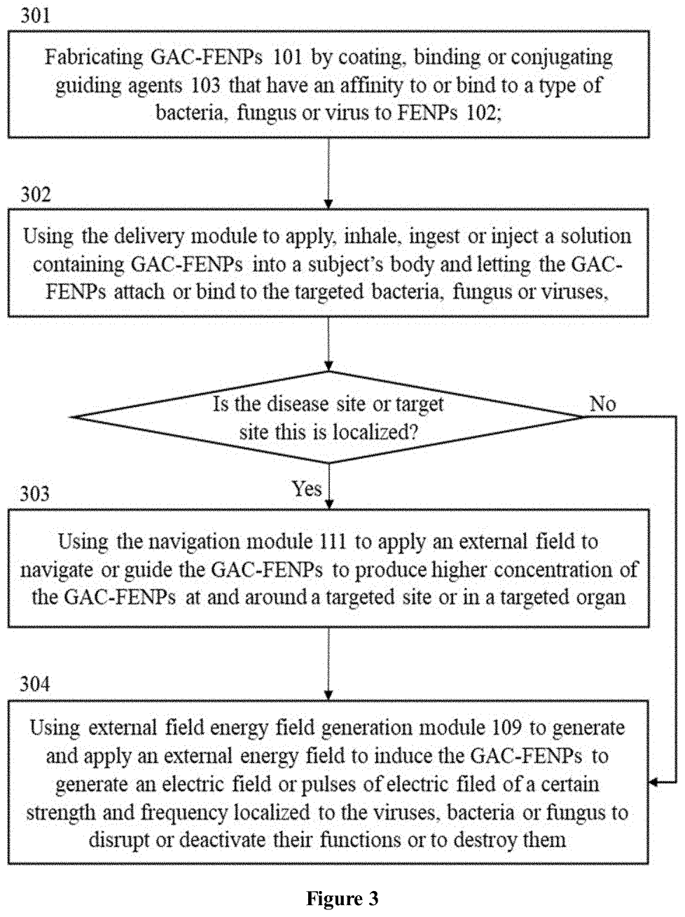

The steps of another preferred embodiment of applying the apparatus for the intended purpose is shown in FIG. 3, comprising:

Step 1 (301): Fabricating GAC-FENPs 101 by coating, binding or conjugating guiding agents 103, such as antibodies or protein, that have an affinity to or bind to a type of bacteria, fungus or virus to FENPs 102;

Step 2 (302): Using the delivery module, e.g., 105 or 107, to apply, inhale, ingest or inject a solution containing GAC-FENPs into a subject's body and letting the GAC-FENPs attach or bind to the targeted bacteria, fungus, viruses, or diseased cells, whereas injection may be via subcutaneous (SC), intraperitoneal (IP), or intravenous (IV) injection (including IV injection or dripping using a catheter) of a solution 106 containing GAC-FENPs, ingestion may be via oral intake (OI) or by other means, inhale may be via nasal inhale of a powder or mist form 108 of the GAC-FENPs, and applying may be a topical application, e.g., a cream or patch that is spread or adhered to the skin of the affected area for treating bacteria, fungus or virus infections of the skin, e.g., in treating bacterial acne or infection, or topical fungal or viral infections;

Step 3 (303): Using the navigation module 111 to apply an external field, e.g., a magnetic or electromagnetic field, to navigate or guide the GAC-FENPs to produce higher concentration of the GAC-FENPs at and around a targeted site or in a targeted organ, e.g., lungs in the case of a virus, fungus or bacteria that attacks the respiratory system or body part with a high concentration of the targeted bacteria, fungus, viruses, or diseased cells. This step is optional and applicable to a disease site or target site this is localized, e.g., the site of infection or attack by the bacteria, fungus, viruses, or diseased cells, and is skipped and not or less applicable when the bacteria, fungus, viruses, or diseased cells are widely distributed, e.g., in the circulatory system; and

Step 4 (304): Using external field energy field generation module 109 to generate and apply an external energy field, e.g., a magnetic, electromagnetic or ultrasonic energy field to induce the GAC-FENPs to generate an electric field or pulses of electric filed of a certain strength and frequency localized to the viruses, bacteria or fungus to disrupt or deactivate their functions or to destroy them. For a bacteria, fungus or virus infection that is localized, the external field energy field generation module 109 is programmed or controlled by the control module 110 to produce or apply a localized external energy field this is concentrated to or stronger at the disease site. For a disease in which the targeted bacteria, fungus, viruses, or diseased cells are widely distributed, the external field energy field generation module 109 is programmed or controlled by the control module 110 to produce or apply a wide-area external energy field that covers a large body area or the whole or most part of the body so that bacteria, fungus, viruses, or diseased cells that are circulating in the body can be disrupted or destroyed.

In one embodiment, the strength and/or frequency of the external energy field in Step 4 is chosen to cause the GAC-FENPs bound to or in close proximity to targeted bacteria, fungus, viruses, or diseased cells. In such embodiments, the control module 110 may integrate or contain the function to program and control the navigation module 111 as well to disrupt or destroy them but does not cause other GAC-MENPs that still remain in the body and unbound to targeted bacteria, fungus, viruses, or diseased cells to harm healthy or untargeted cells. In another embodiment, a sufficiently long waiting period is inserted between Steps 2 and 4 to give the body sufficient time to excrete most or all of the free GAC-FENPs that did not bind to bacteria, fungus, viruses, or diseased cells out of the body. This reduces the risk of GAC-FENPs damaging or destroying healthy or untargeted cells and gives more freedom in selecting the strength and/or frequency of the external energy field in Step 4 to disrupt or destroy the bacteria, fungus or virus.

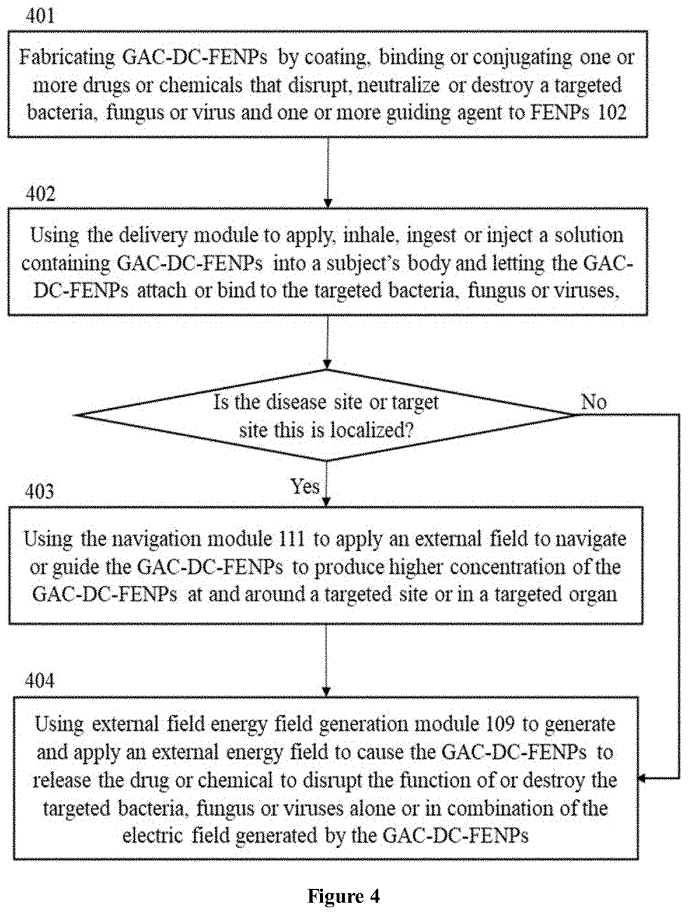

Drugs or chemicals coated, bound or conjugated to MENPs can be released by applying an external magnetic field. Another embodiment of the apparatus comprises FENP nanoparticles that are coated, bounded, conjugated or functionalized with both one or more drugs, molecules or chemicals that modify, affect or neutralize a targeted bacteria, fungus or virus and one or more guiding agent to produce Guide-Agent-Conjugated Drug-Coated FENPs (GAC-DC-FENPs). The steps of a preferred embodiment of applying the apparatus with the GAC-DC-FENPs for the intended purpose is shown in FIG. 4, comprising:

401: Fabricating Guide-Agent-Conjugated Drug-Coated FENPs (GAC-DC-FENPs) by coating, binding or conjugating one or more drugs or chemicals that disrupt, neutralize or destroy a targeted bacteria, fungus or virus and one or more guiding agent to FENPs 102;

402: Using the delivery module, e.g., 105 or 107, to apply, inhale, ingest or inject a solution containing GAC-DC-FENPs into a subject's body and letting the GAC-DC-FENPs attach or bind to the targeted bacteria, fungus, viruses, or diseased cells, whereas injection may be via subcutaneous (SC), intraperitoneal (IP), or intravenous (IV) injection (including IV injection or dripping using a catheter) of a solution containing GAC-DC-FENPs, ingestion may be via oral intake (0I) or by other means, inhale may be via nasal inhale of a powder or mist form of the GAC-DC-FENPs, and applying may be a topical application, e.g., a cream or patch that is spread or adhered to the skin of the affected area for treating bacteria, fungus or virus infections of the skin, e.g., in treating bacterial skin infection or acne, topical fungal or viral infections;

403: Using the navigation module 111 to apply an external field, e.g., a magnetic or electromagnetic field, to navigate or guide the GAC-DC-FENPs to produce higher concentration of the GAC-DC-FENPs at and around a targeted site or in a targeted organ, e.g., lungs in the case of a virus that attacks the respiratory system or body part with a high concentration of the targeted bacteria, fungus, viruses, or diseased cells. This step is optional and applicable to a disease site this is localized, e.g., the site of infection or attack by the bacteria, fungus, viruses, or diseased cells, and is skipped and not or less applicable when the bacteria, fungus, viruses, or diseased cells are widely distributed, e.g., in the circulatory system; and

404: Using external field energy field generation module 109 to generate and apply an external energy field, e.g., a magnetic, electromagnetic or ultrasonic energy field to cause the GAC-DC-FENPs to release the drug or chemical to disrupt the function of or destroy the targeted bacteria, fungus, viruses, or diseased cells alone or in combination of the electric field generated by the GAC-DC-FENPs. For a bacteria, fungus or virus infection that is localized, the external field energy field generation module 109 is programmed or controlled by the control module 110 to produce or apply a localized external energy field this is concentrated to or stronger at the disease site to release the chemical or drug there. For a disease in which the targeted bacteria, fungus, viruses, or diseased cells are widely distributed, the external field energy field generation module 109 is programmed or controlled by the control module 110 to produce or apply a wide-area external energy field that covers a large body area or the whole or most part of the body so that the chemical or drug is released in the large body area or the whole or most part of the body to tackle the bacteria, fungus, viruses, or diseased cells that are circulating in the body. The external energy field, e.g., magnetic or electromagnetic field, for releasing the chemical or drug can be constant, e.g., a magnetic field produced by one or more permanent magnets, or alternating, e.g., an electromagnetic field produced by electromagnets driven by varying electric currents under the control of a microcontroller or other analog or digital control circuits, or an ultrasonic energy field produced by an ultrasonic transducer or a beam-forming transducer array.

In an embodiment, GAC-FENPs or GAC-DC-FENPs is used topically for disrupting or destroying bacteria, fungus, viruses, or diseased cells on the surface of skins or in skin pores, e.g., in treating bacterial skin infections or acne, or fungus or viral skin infections. A solution of GAC-FENPs or GAC-DC-FENPs is applied to the affected skin area, a DC energy field e.g., a constant magnetic field, or a DC and an AC energy field e.g., a DC magnetic field and an AC electromagnetic field, can be applied to disrupt or destroy the bacteria, fungus, viruses, or diseased cells using the induced electric field and/or the released chemical or drug.

A removal module may be added to the apparatus to be used to remove the MENPs from the subject's body. The removal module 112 as shown in FIG. 1B produces a sufficiently strong energy field to the areas of the subject's body that contain GAC-FENPs to attract the GAC-FENPs to facilitate the removal of the GAC-FENPs from the subject's body. The removal module 112 applies an extraction solution that contains agents that binds to the GAC-FENPs; attracts the GAC-FENPs bound the agents to the surface of the subject's body for removal. The removal module may be either an independent module or an attachment or component of the external field generation module 109, especially when the energy field used to remove the GAC-FENPs are the same type of field used to generate electric field on the GAC-FENPs. In such embodiments, the same energy field generation mechanism in the external field generation module 109 is used but the control module 110 in 109 programs or controls 109 to produce the spatial, temporal or spatiotemporal pattern of the generated energy field to achieve the goal of removing the GAC-FENPs or guide the GAC-FENPs to location(s) that facilitate the removal or excretion of the GAC-FENPs out of the body.

Techniques and technologies may be described herein in terms of functional and/or logical block components, and with reference to symbolic representations of operations, processing tasks, and functions that may be performed by various computing components or devices. Such operations, tasks, and functions are sometimes referred to as being computer-executed, computerized, processor-executed, software-implemented, or computer-implemented. It should be appreciated that some block components shown in the figures (e.g. control module 8 or 110) may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software or firmware, various elements of the systems described herein are essentially the code segments or executable instructions that, when executed by one or more processor devices, cause the host computing system to perform the various tasks. In certain embodiments, the program or code segments are stored in a tangible processor-readable medium, which may include any medium that can store or transfer information. Examples of suitable forms of non-transitory and processor-readable media include an electronic circuit, a semiconductor memory device, a ROM, a flash memory, an erasable ROM (EROM), a floppy diskette, a CD-ROM, an optical disk, a hard disk, or the like.

In the foregoing specification, embodiments of the present invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicant to be the scope of the invention, is the literal and equivalent scope of the set of claims that issue from this application, in the specific form in which such claims issue, including any subsequent correction.

1. An antimicrobial apparatus for disrupting, deactivating or destroying microorganism such as bacteria, fungus, viruses, or diseased cells comprising:
(i) Guiding-Agent-Conjugated Field-Electric Nanoparticles (GAC-FENPs) that are produced by coating, binding, functionalizing or conjugating to Field-Electric Nano-Particles (FENPs) with one or more guiding agent that specifically targets or binds to a targeted bacteria, fungus, virus, or diseased cells;
(ii) a delivery module that delivers the GAC-FENPs into a subject's body; and
(iii) an external energy field generation module that generates and applies an external energy field to act on the GAC-FENPs after the GAC-FENPs are in the proximity of, or bind to, the targeted bacteria, fungus, viruses, or diseased cells and produce a localized electric field or pulses of electric field that acts or act on the targeted bacteria, fungus, viruses, or diseased cells to disrupt or deactivate the function of, or destroy, the targeted bacteria, fungus, viruses, or diseased cells.

2. The antimicrobial apparatus according to claim 1, wherein the one or more guiding agent comprises one or more of immunoglobulin, antibody, antibody mimetic, cell adhesion peptides, ligands, or other molecules or proteins that have an affinity to or bind to a targeted bacteria, fungus or virus.

3. The antimicrobial apparatus according to claim 1, wherein the external energy field generation module comprises a magnetic field or electromagnetic field generation module, the Field-Electric Nano-Particles (FENPs) comprise Magneto-Electric Nano-Particles (MENPs), and the GAC-FENPs comprise Guiding-Agent-Conjugated Magneto-Electric Nanoparticles (GAC-MENPs); and
wherein the external energy field generation module generates a magnetic or an electromagnetic field and applies the magnetic or the electromagnetic field to act on the GAC-MENPs.

4. The antimicrobial apparatus according to claim 3, wherein the MENPs each has a core-shell structure, or it comprises a composition of CoFe₂O₄—BaTiO₃, NiFe₂O₄—BaTiO₃, or Fe₃O₄—BaTiO₃.

5. The antimicrobial apparatus according to claim 3, wherein the external energy field generation module comprises one or more stationary or moving permanent magnets, one or more electromagnets driven by varying electric currents, or any combination thereof.

6. The antimicrobial apparatus according to claim 3, wherein the magnetic or electromagnetic field generation module further applies one or more of the following mechanisms to disrupt or destroy the bacteria, fungus or virus after the GAC-FENPs have bound to or have electroporated inside the targeted bacteria, fungus or virus:
applying an alternating external magnetic field to induce an alternating electric field on the GAC-MENPs whereas the strength and frequency of the field is selected such that it disrupts the functions of the bacteria, fungus, viruses, or diseased cells;
applying an alternating external magnetic field to induce mechanical motions of the GAC-MENPs to disrupt or deactivate the functions or to physically damage the bacteria, fungus, viruses, or diseased cells, wherein the mechanical motions may include linear motion, slicing, collisions or vibrations, or combinations thereof;
applying an alternating external magnetic field of a resonant frequency that is modified by the interaction of GAC-MENPs with the nano-environment to induce a ferromagnetic resonance in the GAC-MENPs to disrupt or deactivate or shut down the operation of the bacteria, fungus, viruses, or diseased cells; or,
applying an external constant magnetic field to modify the resonant frequency of the GAC-MENPs, and thereafter applying an alternating external magnetic field at a frequency that causes a ferromagnetic resonance in the GAC-MENPs at or near the modified resonant frequency to selectively disrupt or deactivate the function of the bacteria, fungus, viruses, or diseased cells.

7. The antimicrobial apparatus according to claim 3 wherein the external energy field generation module generates an electromagnetic field in one or more of the following frequency ranges from 1 Hz to 10 trillion Hz (THz), such as audio frequency, radio frequency, optical frequency, near or far infrared frequency.

8. The antimicrobial apparatus according to claim 3, further comprising a navigation module comprising one or more permanent magnets or electro-magnets that are placed, injected or implanted in or near a site with a high concentration of the (or a concentrated) targeted virus, bacteria, fungus or diseased cells to attract or guide the GAC-MENPs to said site.

9. The antimicrobial apparatus according to claim 3, wherein the external field energy field generation module comprises a control module that programs or controls the external field energy field generation module to produce or apply external energy field with a desired spatial, temporal or spatiotemporal pattern to yield a desired treatment effect or plan.

10. The antimicrobial apparatus according to claim 1, further comprising a navigation module that generates an external energy field and uses it to guide, attract and/or enhance accumulation of the GAC-FENPs into a site with a high concentration of targeted viruses, bacteria, fungus or diseased cells.

11. The antimicrobial apparatus according to claim 1, wherein the external energy field generation module comprises an ultrasound energy field generation module, the Field-Electric Nano-Particles (FENPs) comprise Ultrasonic-Electric Nano-Particles (UENPs), and the GAC-FENPs comprise Guiding-Agent-Conjugated Ultrasonic-Electric Nanoparticles (GAC-UENPs); and
wherein the external energy field generation module generates an ultrasound energy field and applies the ultrasound energy field to act on the GAC-UENPs.

12. The antimicrobial apparatus according to claim 1, wherein the delivery module comprises an injection syringe for injecting a solution of the GAC-FENPs into a subject's body, an aerosolization device for inhalation of the GAC-FENPs into a subject's body, a pressured or diffusion delivery device for infuse the GAC-FENPs into a subject's body across the skin, or an encapsulation device to produce GAC-FENPs containing capsules or pills for ingesting the GAC-FENPs into a subject's body.

13. The antimicrobial apparatus according to claim 1, wherein the external energy field generation module is configured to adjust the characteristics of the generated external field so that it causes electric field on the GAC-FENPs bound to the targeted bacteria, fungus, viruses, or diseased cells to disrupt or deactivate or damage the targeted bacteria, fungus, viruses, or diseased cells but it does not cause other GAC-FENPs that still remain in the body but unbound to targeted bacteria, fungus, viruses, or diseased cells to harm healthy or untargeted cells.

14. The antimicrobial apparatus according to claim 1, wherein the FENPs' surfaces are first coated, functionalized or conjugated with one or more layer of material, molecule or compound to modify its surface property before being coated, conjugating or functionalized with the guiding agent that targets or binds to a targeted bacterium or virus to produce the GAC-FENPs.

15. The antimicrobial apparatus according to claim 1, which is used for localized treatment of a targeted site, wherein the external energy field generation module is configured to generate and apply a first external energy field to lead to a higher concentration of GAC-FENPs at and around a targeted site or in an organ or body part with a high concentration of the targeted bacteria, fungus, viruses, or diseased cells before it generates and applies a second external energy field to cause the GAC-FENPs to generate an electric field or pulses of electric field to disrupt the function or destroy the targeted bacteria, fungus, viruses, or diseased cells.

16. The antimicrobial apparatus according to claim 1, wherein the external energy field generation module is configured to generate and apply an energy field after a waiting period to give the subject's body sufficient time to excrete most or all of the free GAC-FENPs that did not bind to targeted bacteria, fungus, viruses, or diseased cells out of the body, and/or for the GAC-FENPs to target or bind to the targeted viruses, bacteria or fungus before generating and applying the external energy field to cause damages to the targeted bacteria, fungus, viruses, or diseased cells.

17. The antimicrobial apparatus according to claim 1, wherein the FENPs are coated, bounded, conjugated or functionalized both with one or more drugs, molecules or chemicals that modify, affect or neutralize a targeted bacteria, fungus, virus or diseased cells and one or more guiding agents that target a bacteria, fungus or virus to produce Guiding-Agent-Conjugated Drug-Coated FENPs (GAC-DC-FENPs).

18. The antimicrobial apparatus according to claim 1, further comprising a removal module configured for removing the FENPs from the subject's body.

19. The antimicrobial apparatus according to claim 18, wherein the removal module is configured to produce a sufficiently strong energy field and apply it to areas of the subject's body that contain GAC-FENPs to attract the GAC-FENPs to facilitate the removal of the GAC-FENPs from the subject's body.

20. The antimicrobial apparatus according to claim 18, wherein the removal module is configured to apply an extraction solution that contains agents that binds to the GAC-FENPs; and attract the GAC-FENPs bound with the agents to the surface of the subject's body for removal.

21. The antimicrobial apparatus according to claim 1, wherein the FENPs are coated, bounded, conjugated or functionalized with one or more drugs, molecules or chemicals (instead of said one or more guiding agents) that modify, affect or neutralize a targeted bacteria, fungus or virus to produce Drug-Coated FENPs (DC-FENPs).
